(19) United States
(12) Patent Application Publication
(10) Pub. No.: US 2009/0179547 A1
(43) Pub. Date: Jul. 16, 2009

(54) FLAT UV LAMP WITH COPLANAR DISCHARGE AND USES THEREOF

(75) Inventors: Guillaume Auday, Bussiere St. Geoges (FR); Jingwei Zhang, Massy (FR); Didier Duron, Boulogne Billancourt (FR)

Correspondence Address: OBLON, SPIVAK, MCCLELLAND MAIER & NEUSTADT, P.C., 1940 DUKE STREET, ALEXANDRIA, VA, 22314, US

(73) Assignee: SAINT-GOBAIN GLASS FRANCE, Courbevoie (FR)

(21) Appl. No.: 12/064,153

(22) PCT Filed: Aug. 10, 2006
(86) PCT No.: PCT/FR2006/050798

§ 371 Date: Oct. 3, 2008

(30) Foreign Application Priority Data
Aug. 19, 2005 (FR) 0552540

(51) Int. Cl. H01J 1/62; H01J 17/00

(52) U.S. Cl. 313/485; 313/581

(57) ABSTRACT

A flat lamp transmitting radiation in UV (ultraviolet), including first and second flat or substantially flat glass elements substantially parallel to each other and defining an internal space filled with gas capable of emitting the radiation in the UV or of exciting a phosphor material possibly present and emitting the radiation in the UV. The phosphor material can be deposited on one face of the first and/or second glass element, the first and/or second glass element being made of a material transmitting the UV radiation. A plurality of pairs of electrodes are capable of being at different potentials and of being supplied by an AC voltage, the pairs associated with the first glass element and placed outside the internal space, the electrodes being in a form of bands and/or wires in the first glass element or in another dielectric element associated with the first glass element.

The present invention relates to the field of flat ultraviolet (UV) lamps and in particular it relates to coplanar-discharge flat UV lamps and to the uses of such lamps.

Conventional UV lamps are formed by UV fluorescent tubes filled with mercury and placed side by side in order to form an emitting surface. These tubes have a limited lifetime. Furthermore, the uniformity of the UV radiation emitted is difficult to obtain for large areas. Finally, such lamps are heavy and bulky.

Document U.S. Pat. No. 5,006,758 proposes a flat UV tanning lamp consisting of two glass plates that transmit UVA, said plates being kept a short distance apart and hermetically sealed so as to contain a gas under reduced pressure. An electrical discharge produces UV radiation, which excites a phosphor coating emitting in the UVA.

One of the glass plates bears the phosphor coating on its internal face and the other glass plate bears, on its internal face, series of conducting coatings or electrodes constituting a cathode and an anode at a given instant. The discharge generated between anode and cathode is called a coplanar discharge, that is to say formed along a direction hugging the main surface of the glass plate.

The electrodes are protected by a dielectric coating intended, by capacitively limiting the current, to avoid loss of material of the electrodes by ion bombardment near the glass plate. For effective protection of the electrodes, it is essential to choose a sufficiently resistant dielectric.

At the same time, it is essential to control the uniformity of the dielectric and its homogeneity, for example to avoid the presence of bubbles, to avoid arcs and to obtain satisfactory optical performance.

Furthermore, this dielectric layer requires an additional manufacturing step incurring an additional cost, making the UV lamp designed only for high value-added applications.

Finally, the reliability of the UV lamp is difficult to obtain, its UV emission properties varying from one lamp to another and furthermore requiring their capacitances to be tested.

Document JP 2004152534 describes a flat lamp emitting in the UV with a glass element that transmits the UV, a dielectric element and a pair of electrodes placed on the external face of the glass element and as far away as possible from each other in order to let through the UV radiation. This lamp is not effective for all gases.

Document U.S. Pat. No. 6,049,086 proposes a flat lamp emitting in the UV with a glass element and a dielectric element with pairs of electrodes on its external face, each of the electrodes being a wire in a dielectric tube. This lamp is complex.

The object of the invention is to provide a flat UV lamp that is reliable, of high performance, of simpler design and rapid and/or easy to manufacture.

For this purpose, the invention proposes a flat lamp transmitting radiation in the UV (Ultraviolet), comprising:
first and second flat or substantially flat glass elements kept substantially parallel to each other and defining an internal space filled with gas capable of emitting said radiation in the UV or of exciting a phosphor material possibly present and emitting said radiation in the UV, said phosphor material then being deposited on one face of the first and/or second glass element, the first and/or second element being made of a material transmitting said UV radiation; and
a plurality of pairs of electrodes capable of being at different potentials and of being supplied by an AC voltage, said pairs being associated with the first glass element and placed outside the internal space, the electrodes being in the form of bands and/or wires in the first glass element or in another dielectric element associated with the first glass element.

Electrodes in the form of bands and/or in a dielectric element (glass and/or plastic for example) are simple to produce and the plurality of electrodes guarantees satisfactory luminous efficiency for all gases. Preferably, most if not all of the electrodes may be of the same design.

The choice of the two glass elements simplifies the mounting of the lamp and guarantees a strong and durable flat lamp. The first glass element may be chosen so as to transmit or absorb the UV depending on the desired applications or configurations (emission by the two glass elements through the electrodes, etc.), thus giving a freedom of choice.

With external electrodes in the form of bands or integrated electrodes, the first glass element acts as a capacitive protection for the electrodes against ion bombardment, and consequently forms a dielectric of constant thickness and excellent uniformity, guaranteeing uniformity of the UV radiation emitted by the lamp.

By placing the electrodes on the outside of the enclosure under reduced pressure of plasma gas, this structure makes it possible for the manufacturing cost of the UV lamp to be considerably lowered. The manufacture of the UV lamp is also simplified and made reliable, by eliminating manufacturing errors.

Furthermore, the problem of connecting the power supply is solved much more simply than in the case of known systems in which the electrical connectors must pass through the hermetically sealed gas-containing enclosure.

The UV lamp may have dimensions of the order of those currently achieved with fluorescent tubes, or even larger, for example with an area of at least 1 m².
Preferably, the transmission factor of the lamp according to the invention about the peak of said UV radiation is equal to 50% or higher, more preferably equal to 70% or higher, and even 80% or higher.

In a lamp configuration with only one face of a glass element transmitting the UV, the other glass element may be opaque, for example a glass-ceramic, or even a non-glass dielectric.

However, the translucent character may serve to position the lamp or for displaying or verifying the operation of the lamp.

In a preferred embodiment, the electrodes are covered or integrated at least partly in a dielectric element, preferably flat and/or common to all the electrodes, chosen from the first glass element, another glass element (therefore forming a reinforced glass) and/or at least one plastic, or possibly a glass or plastic element associated with a gas-filled cavity.

For this dielectric element, the uniformity or homogeneity requirements are no longer crucial. It is also possible to choose from a very wide range of dielectrics and geometries. Furthermore, if it is desired to have a lamp emitting via both sides, it is easier to choose a UV-transmitting dielectric.

This element may form part of an insulating, vacuum or argon-filled glazing unit, or a glazing unit with a single air cavity. A simple varnish of sufficient thickness (where appropriate in order to absorb the UV radiation) may also be used.

This dielectric element serves as mechanical or chemical protection and/or forms a lamination interlayer and/or provides satisfactory electrical isolation should it be required, for example if this face bearing the electrodes is easily accessible.

Thus, the electrodes may be associated with the first glass element in various ways: they may for example be incorporated into the latter or into a common dielectric element, or, when they are in the form of bands, they may be directly deposited on its external face or on a bearing element (corresponding to said dielectric element), this bearing element being joined to the first glass element in such a way that the electrodes are pressed against its external face.

The electrodes may also be sandwiched between a first dielectric and a second dielectric, the assembly being joined to the first glass element.

In a first example, the first dielectric is a lamination interlayer and the second dielectric is a back glass plate or a rigid plastic, preferably one that is transparent. As a variant, the electrodes may be placed between said first glass element and the lamination interlayer.

In a second example, the electrodes are on a preferably thin and/or transparent dielectric located between two lamination interlayers, the dielectric being for example a plastic film or a thin glass sheet.

These first and second dielectric elements may therefore be formed in various combinations, by combining a glass element and a plastic element (whether rigid, monolithic or laminated) and/or plastic films or other resins capable of being assembled by adhesive bonding with glass products.

Suitable plastics are, for example:
polyurethane (PU) used soft, an ethylene/vinyl acetate copolymer (EVA) or polyvinyl butyral (PVB), these plastics serving as lamination interlayer, for example with a thickness between 0.2 mm and 1.1 mm, especially between 0.3 and 0.7 mm, optionally incorporating the electrodes in their thickness or carrying the electrodes; and
rigid polyurethane, polycarbonates, acrylates, such as polymethyl methacrylate (PMMA), used especially as rigid plastic, and optionally carrying electrodes.

It is also possible to use PE, PEN or PVC, or polyethylene terephthalate (PET), the latter possibly being thin, especially between 10 and 100 μm in thickness, and possibly bearing the electrodes.

Where appropriate, it is necessary to ensure, of course, compatibility between various plastics used, especially as regards their good adhesion.

Of course, any aforementioned dielectric element is chosen to be substantially transparent to said UV radiation if it is placed on the emission side of the UV lamp.

The electrodes in band form may be linear or of more complex, nonlinear, shape, for example angled, V-shaped, corrugated or zigzagged, the spacing between electrodes being kept substantially constant. For example, the electrodes may be in the form of interpenetrated combs with a constant spacing between adjacent teeth.

In one advantageous embodiment, the electrodes are based on a material transmitting said UV radiation or are arranged and/or adapted so as to allow overall transmission to said UV radiation (if the material absorbs or reflects UV) and the first element is made of said material transmitting said UV radiation.

The electrode material transmitting said UV radiation may be a very thin film of gold, for example with a thickness of around 10 nm, or of alkali metals, such as potassium, rubidium, cesium, lithium or potassium for example with a thickness of 0.1 to 1 μm, or else an alloy, for example a 25% sodium/75% potassium alloy.
In the latter embodiment, it is possible to choose a second glass element that absorbs said UV radiation for a UV lamp having only a single face transmitting the UV.

In the latter embodiment, the electrodes may be substantially parallel bands having a width I1 and being spaced apart by a distance d1, the ratio of I1 to d1 possibly being between 10% and 50%, in order to allow an overall UV transmission of at least 50% on one side of the electrodes, the I1/d1 ratio also possibly being adjusted according to the transmission of the associated glass element.

An electrode material relatively opaque to said UV radiation is for example fluorine-doped tin oxide (SnO₂:F), mixed indium tin oxide (ITO), silver, copper or aluminum.

Alternatively, if the UV radiation is transmitted only on the second glass element side, it does not matter what the ratio of I1 to d1 is.

The electrodes in band form may be solid electrodes, especially formed from contiguous conducting wires (parallel wires, braided wires, etc.) or from a ribbon (made of copper, to be bonded, etc.) or from a coating deposited by any means known to those skilled in the art, such as liquid deposition, vacuum deposition (magnetron sputtering, evaporation), by pyrolysis (powder or gas) or by screen printing.

To form bands in particular, it is possible to employ masking systems in order to obtain the desired distribution directly, or else to etch a uniform coating by laser ablation or by chemical or mechanical etching.

Electrodes may also each be in the form of an array of essentially elongate conducting features, such as conducting lines (likened to very narrow bands) or actual conducting wires. These features may be substantially straight or corrugated, zigzagged, etc.

This array may be defined by a given pitch p1 (the minimum pitch in the case of a plurality of pitches) between features and a width I2 of features (the maximum width in the case of a plurality of widths). Two series of features may be crossed. This array may especially be organized like a grid, fabric or cloth. These features are for example made of metal, for example tungsten, copper or nickel.

Thus, it is possible to obtain overall UV transparency by adapting the ratio of I1 to d1 according to the desired transparency, as already described and/or using the array of conducting features and adapting the width I2 and/or the pitch p1 according to the desired transparency.

Thus, the ratio of the width I2 to the pitch p1 may preferably be equal to 50% or less, preferably 10% or less and even more preferably 1% or less.

For example, the pitch p1 may be between 5 μm and 2 cm, preferably between 50 μm and 1.5 cm and even more preferably between 100 μm and 1 cm, and the width I2 may be between 1 μm and 1 mm, preferably between 10 and 50 μm.

As examples, it is possible to use a conducting array (grid, etc.) on a plastic sheet, for example of the PET type, with a pitch p1 of between 100 μm and 300 μm and a width I2 of 10 to 20 μm, or else an array of conducting wires at least partly integrated into a lamination interlayer, especially made of PVB or PU, with a pitch p1 between 1 and 10 mm, especially 3 mm, and a width I2 between 10 and 50 μm, especially between 20 and 30 μm.

The lamp may comprise a material that reflects said UV radiation and partially or entirely covering one face of the first or second glass element, for example made of aluminum.

In a first configuration in which the UV radiation is transmitted via the first glass element, this material preferably coats the internal face of the second glass element.

In a second configuration in which the UV radiation is transmitted via the second glass element, the electrodes themselves may be made of said reflecting material.

The material transmitting said UV radiation may be preferably chosen from quartz, silica, magnesium fluoride (MgF₂) or calcium fluoride (CaF₂), a borosilicate glass or a glass containing less than 0.05% Fe₂O₃.

To give examples, for thicknesses of 3 mm:
magnesium or calcium fluorides transmit more than 80%, or even 90%, over the entire range of UV bands, that is to say UVA (between 315 and 380 nm), UVB (between about 280 and 315 nm), UVC (between 200 and 280 nm) and VUV (between about 10 and 200 nm);
quartz and certain high-purity silicas transmit more than 80%, or even 90%, over the entire range of UVA, UVB and UVC bands;
borosilicate glass, such as Borofloat from Schott, transmits more than 70% over the entire UVA band; and

soda-lime-silica glass with less than 0.05% Fe(III) or Fe₂O₃, especially the glass Diamant® from Saint-Gobain, the glass Optiwhite® from Pilkington, and the glass B270 from Schott, transmits more than 70% or even 80% over the entire UVA band.

However, a soda-lime-silica glass, such as the glass Planilux® sold by Saint-Gobain, has a transmission of more than 80% above 360 nm, which may be sufficient for certain constructions and certain applications.

In the structure of the flat lamp according to the invention, the gas pressure in the internal space may be around 0.05 to 1 bar. A gas or a gas mixture is used, for example a gas that efficiently emits said UV radiation, especially xenon, or mercury or halides, and an easily ionizable gas capable of forming a plasma (plasma gas), such as a rare gas like neon, xenon or argon or even helium, or halogens, or even air or nitrogen.

The halogen content (when the halogen is mixed with one or more rare gases) is chosen to be less than 10%, for example 4%. It is also possible to use halogenated compounds. The rare gases and the halogens have the advantage of being insensitive to the environmental conditions.

Table 1 below indicates the radiation peaks of the particularly effective UV-emitting gases.

TABLE 1

| UV-emitting gas(es) | Peak(s) (in nm) |
|---|---|
| Xe | 172 |
| F₂ | 158 |
| Br₂ | 269 |
| C | 259 |
| I₂ | 342 |
| XeI/KrI | 253 |
| ArBr/KrBr/XeBr | 308/207/283 |
| ArF/KrF/XeF | 351/249/351 |
| ArCl/KrCl/XeCl | 351/222/308 |
| Hg | 185, 254, 310, 366 |

According to one feature of the invention, the phosphor material forms a coating on an internal face of the first element or on one face (either the internal or external face) of the second glass element.

There are in particular phosphors that emit in the UVC from exposure to VUV radiation. For example, UV radiation at 250 nm is emitted by phosphors after being excited by VUV radiation shorter than 200 nm, such as from mercury or a rare gas.

There are also phosphors that emit in the UVA or near UVB when exposed to VUV radiation. Mention may be made of gadolinium-doped materials such as YBO₃:Gd; YB₂O₅:Gd; LaP₃O₉:Gd; NaGdSiO₄; YAl₃(BO₃)₄:Gd; YPO₄:Gd; YAlO₃:Gd; SrB₄O₇: Gd; LaPO₄:Gd; LaMgB₅O₁₀: Gd, Pr; LaB₃O₈: Gd, Pr; and (CaZn)₃(PO₄)₂:Tl.

There are also phosphors that emit in the UVA when exposed to UVC radiation. Mention may for example be made of LaPO₄:Ce; (Mg,Ba)Al₁₁O₁₉:Ce; BaSi₂O₅:Pb; YPO₄:Ce; (Ba,Sr,Mg)₃Si₂O₇:Pb and SrB₄O₇:Eu.

For example, UV radiation above 300 nm, especially between 318 nm and 380 nm, is emitted by phosphors after being excited by UVC radiation of around 250 nm.

Furthermore, it may be advantageous to incorporate a coating having a given functionality into the UV lamp according to the invention. This may be an anti-soiling or self-cleaning coating, especially a TiO₂ photocatalytic coating deposited on the glass element opposite the emitting face, this coating possibly being activated by UV radiation.

The lamp may comprise a coating made of another phosphor material that emits in the visible, associated with the second glass element and placed in a limited region (on the internal and/or external face) of this second element. This region may optionally constitute decorative features or constitute a display, such as a logo or a trademark or an indicator of the operating status of the lamp.

The glass elements may be of any shape—the outline of the elements may be polygonal, concave or convex, especially square or rectangular, or curved, especially round or oval.

The glass elements may be slightly curved, with the same radius of curvature, and are preferably kept a constant distance apart, for example by spacers, such as glass beads. These spacers, which may be termed discrete spacers when their dimensions are considerably smaller than the dimensions of the glass elements, may take various forms, especially in the form of spheres, parallel-faced bitruncated spheres, cylinders, but also parallelepipeds of polygonal cross section, especially cruciform cross section, as described in document WO 99/56302.

The gap between the two glass elements may be fixed by the spacers so as to have a value of around 0.3 to 5 mm. A technique for depositing the spacers in vacuum insulating glazing units is known from FR-A-2 787 133. According to this process, spots of adhesive are deposited on a glass plate, especially spots of enamel deposited by screen printing, with a diameter equal to or less than the diameter of the spacers, and then the spacers are rolled over the glass plate, which is preferably inclined, so that a single spacer adheres to each spot of adhesive. The second glass plate is then placed on the spacers and the peripheral seal deposited.

The spacers are made of a nonconducting material in order not to participate in the discharges or to cause a short circuit. Preferably, they are made of glass, especially of the soda-lime type. To prevent light loss by absorption in the material of the spacers, it is possible to coat the surface of them with a material that is transparent or reflective in the UV, or with a phosphor material identical to or different from that used for the glass element(s).

According to one embodiment, the lamp may be produced by manufacturing firstly a sealed enclosure in which the intermediate air cavity is at atmospheric pressure, then a vacuum is created and the plasma gas introduced at the desired pressure. According to this embodiment, one of the glass elements includes at least one hole drilled through its thickness and obstructed by a sealing means.

The UV lamp as described above may be used both in the industrial sector, for example in the beauty, biomedical, electronics or food fields, and in the domestic sector, for example for decontaminating air or tap water, drinking water or swimming pool water, for UV drying and for polymerization.

By choosing radiation in the UVA or even in the UVB, the UV lamp as described above may be used:
as a tanning lamp (especially 99.3% in the UVA and 0.7% in the UVB according to the standards in force);
for dermatological treatments (especially radiation in the UVA at 308 nm);
for photochemical activation processes, for example for polymerization, especially of adhesives, or crosslinking or for drying paper;
for the activation of fluorescent material, such as ethidium bromide used in gel form, for analyzing nucleic acids or proteins; and

for activating a photocatalytic material, for example for reducing odors in a refrigerator or dirt.

By choosing radiation in the UVB, the lamp promotes the formation of vitamin D in the skin.

By choosing radiation in the UVC, the UV lamp as described above may be used for disinfecting/sterilizing air, water or surfaces, by a germicide effect, especially between 250 nm and 260 nm.

By choosing radiation in the far UVC or preferably in the VUV for ozone production, the UV lamp as described above is used especially for the treatment of surfaces, in particular before the deposition of active films for electronics, computing, optics, semiconductors, etc.

The lamp may for example be integrated into household electrical equipment, such as a refrigerator or kitchen shelf.

Other details and advantageous features of the invention will become apparent on reading the examples of the UV flat lamps illustrated by the following figures:

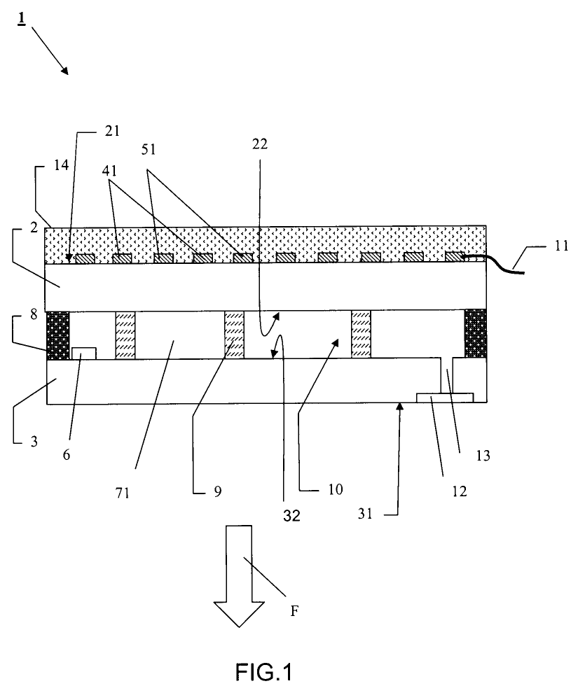

FIG. 1 shows schematically a sectional view of an external coplanar-discharge flat UV lamp in a first embodiment of the invention;

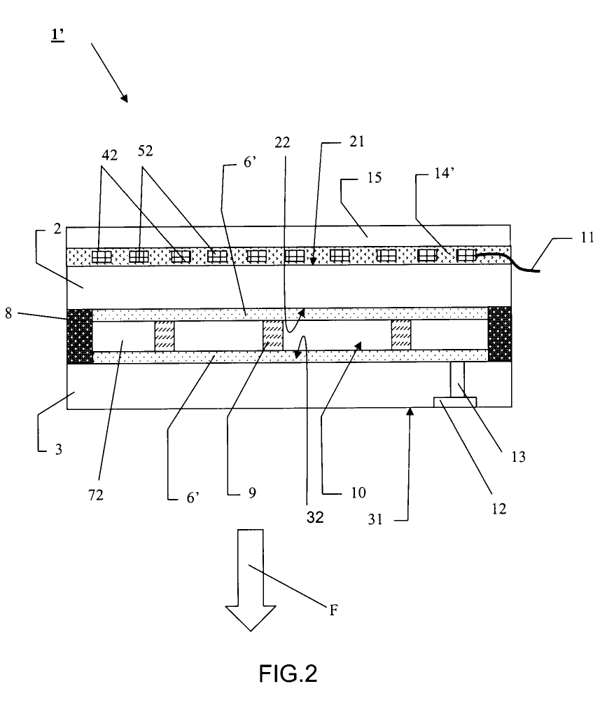

FIG. 2 shows schematically a sectional view of an external coplanar-discharge flat UV lamp in a second embodiment of the invention;

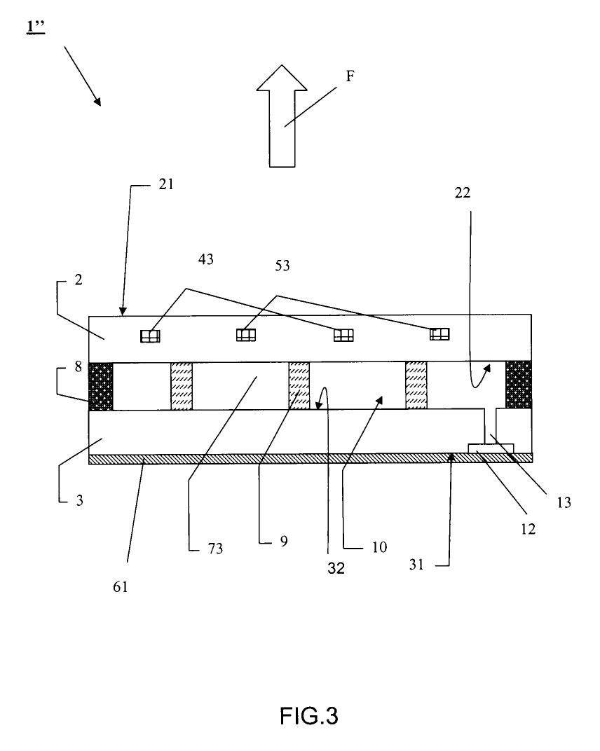

FIG. 3 shows schematically a sectional view of an external coplanar-discharge flat UV lamp in a third embodiment of the invention; and

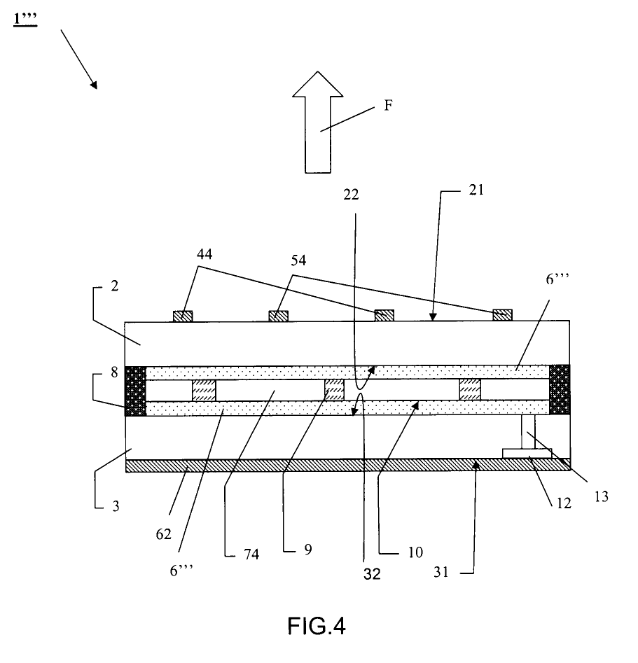

FIG. 4 shows schematically a sectional view of an external coplanar-discharge flat UV lamp in a fourth embodiment of the invention.

It should be pointed out that, for the sake of clarity, the various elements of the objects shown are not necessarily drawn to scale.

FIG. 1 shows a coplanar-discharge flat UV lamp 1 comprising first and second glass plates 2, 3, each having an external face 21, 31 and an internal face 22, 32. The lamp 1 emits UV radiation (indicated symbolically by an arrow F) only via its face 31. This also makes it possible to protect the possibly accessible other side from the UV radiation.

The area of each glass plate 2, 3 is for example about 1 m², or even more, and their thickness is 3 mm.

A plurality of electrodes 41, 51 are coupled in pairs. They are in the form of bands deposited directly on the external face 21, for example screen-printed with silver ink, or else they are adhesively bonded copper bands.

The electrodes could also be bands formed from arrays of conducting wires.

The external face 21 itself—at least in the regions of the electrodes—is fitted with an electrically insulating and protective plastic film 14. In this embodiment, said dielectric 14 may be translucent or opaque, depending on the requirements.

In a variant, the electrodes 41, 51 are placed on this external plastic 14 (or between two plastic films) which is assembled in such a way that the electrodes 41, 51 are pressed against the face 21.

In another variant (not shown), the electrodes are in the glass 2, forming for example a reinforced glass.

The plates 2, 3 are joined together so that their internal faces 22, 32 face each other and are assembled by means of a sealing frit 8, for example a glass frit having a thermal expansion coefficient close to that of the glass plates 2, 3, such as a lead frit.

As a variant, the plates are joined together by an adhesive, for example a silicone adhesive, or else by a heat-sealed glass frame. These sealing modes are preferable if plates 2, 3 having excessively different expansion coefficients are chosen. This is because the first plate 2 may be made entirely of glass material or more generally of dielectric material suitable for this type of lamp, whether transmitting the UV or not and whether translucent or opaque.

The gap between the glass plates is set (generally to a value of less than 5 mm) by glass spacers 9 placed between the plates. Here, the gap is for example between 1 and 2 mm.

The spacers 9 may have a spherical, cylindrical or cubic shape, or they may have another polygonal, for example cruciform, cross section. The spacers may be coated, at least on their lateral surface exposed to the plasma gas atmosphere, with a material that reflects the UV.

The second glass plate 3 has, near the periphery, a hole 13 drilled through its thickness, with a diameter of a few millimeters, the external orifice of which is obstructed by a sealing pad 12, especially made of copper, welded to the external face 31.

A phosphor 6 emitting in the visible is deposited in a limited and peripheral region of the internal face 21—or, in a variant, on the internal face 22 or external face 31—in the form of the letters ‘ON’ in order to indicate the operational state.

The electrodes 41, 51 are supplied via a flexible shim 11 or, as a variant, via a welded wire by a high-frequency voltage signal (not shown), for example with an amplitude of around 1500 V and a frequency between 10 and 100 kHz. More precisely each electrode 41 (electrode 42 respectively) is connected to the same busbar (not shown for the sake of clarity) which is placed on the periphery of the glass plate 2 that is connected to said shim.

Only the electrodes 41 are supplied by the high-frequency signal, the electrodes 51 then being grounded. Alternatively, the electrodes 41 and 51a are supplied, for example, with signals in phase opposition.

Of course, a control system may be provided for varying the voltage, and therefore the illumination.

A coplanar discharge is produced between each pair of electrodes 41, 51.

In the space 10 between the glass plates 2, 3 there is a reduced pressure of 250 mbar of a neon/xenon mixture 71 in order to emit radiation in the VUV. The height of gas may be between 0.5 mm and a few mm in height, for example 2 mm.

At least in the case of the plate 3, high-purity silica will preferably be chosen for low-cost high VUV transmission. Its expansion coefficient is about 54×10⁻⁸ K⁻¹.

This compact and reliable lamp 1 is used for example for the treatment of surfaces, even large ones.

In the embodiment shown in FIG. 2, the structure 1′ of the flat external coplanar-discharge UV lamp has the structure of FIG. 1 except for the elements detailed below.

The plastic film 14 may be replaced with a lamination interlayer 14′ of the PVB or EVA or polyurethane type and a back glass plate 15 (or, as a variant, a polycarbonate or PMMA back plate), thus forming a laminated (composite) glass with the glass plate 2.

The electrodes 42, 52 are bands each formed from an array of conducting wires (for example in the form of a grid, and made of tungsten), which are integrated into the lamination interlayer 14′ with a pitch p1 of 3 mm and a width I2 of about 20 μm.

In a variant, the electrodes 42, 52 are placed on a plastic film, for example a thin PET film, for example with a pitch p1 of 100 μm and a width 12 of 10 μm, located between the lamination interlayer 14′ and another added lamination interlayer.

In another variant, the electrodes 42, 52 are solid, for example placed as a layer on the face 21, especially deposited on the face 21 and produced by etching.

In the space 10 between the plates 2, 3 there is a reduced pressure of 200 mbar of a xenon/indium mixture 72 in order to emit exciting radiation in the UVC.

The internal faces 22, 32 (or, in a variant, the internal face 22 alone, or even the external face with a suitable glass) bear a coating 6′ of phosphor material emitting radiation in the UVA, preferably beyond 350 nm, such as YPO₄:Ce (peak at 357 nm) or (Ba,Sr,Mg)₃Si₂O₇:Pb (peak at 372 nm) or SrB₄O₇:Eu (peak at 386 nm).

At least in the case of the plate 3, and preferably in the case of both plates 2, 3, a soda-lime-silica glass, such as Planilux® sold by Saint-Gobain, is chosen, which gives a UVA transmission at around 350 nm of greater than 80% for low cost. Its expansion coefficient is about 90×10⁻⁸ K⁻¹.

The proposed UVA lamp serves for example as a tanning lamp.

In another variant, a gadolinium-based phosphor is chosen and, at least in the case of the plate 3, a borosilicate glass (for example with an expansion coefficient of about 32×10⁻⁸ K⁻¹) or a soda-lime-silica glass containing less than 0.05% Fe₂O₃, and also a rare gas such as xenon by itself or as a mixture with argon and/or neon.

In the embodiment shown in FIG. 3, the structure of the coplanar-discharge flat UV lamp 1″ has the structure of FIG. 1 except for the elements detailed below.

The lamp 1″ emits UV radiation via its face 21, the plastic 14 being omitted. The electrodes 43, 53 are each in the form of an array of thin conducting wires integrated into the glass 2.

The size of the wires and/or the distance between the wires and/or the width of the electrodes and/or the interelectrode space are adapted accordingly in order to increase the overall UV transmission.

In a variant, the electrodes 43, 53 are screen-printed silver bands deposited on the face 1. This electrode material is relatively opaque to the UV and the ratio of the electrode width I1 to the width of the interelectrode space d1 is therefore adapted accordingly in order to increase the overall UV transmission.

For example, a ratio of the width I1 to the width d1 of the interelectrode space of about 20% or less is chosen, for example the width I1 is equal to 4 mm and the width d1 of the interelectrode space is equal to 2 cm.

In the space 10 between the glass plates 2, 3 there is a reduced pressure of a mixture of rare gases and halogens 73—or of diatomic halogen or even mercury—for UVC radiation preferably between 250 and 260 nm for a germicide effect used in particular for disinfecting/sterilizing air, water or surfaces. For example, mention may be made of Cl₂ or an XeI/KrF mixture.

To let this UVC radiation through the plate 2, this is chosen to be made of fused silica or quartz. The overall transmission with this glass and the electrodes 43, 53 is 80% at 250 nm.

In another variant, a UV-transmitting electrode material is chosen in order to have freedom as regards the structure of the electrodes.

Moreover, the external face 31 (or, in a variant, the internal face 32) bears a coating 61 of UV-reflecting material, for example aluminum, in order to increase the transmission and provide protection from the radiation, irrespective of the dielectric chosen for the plate 3.

In the embodiment shown in FIG. 4, the structure 1′″ of the external coplanar-discharge flat UV lamp has the structure of FIG. 3 except for the elements detailed below.

A Planilux® glass is chosen for the plate 3 and a Planilux® glass with a fluorine-doped tin oxide layer is chosen for the plate 2, which layer is etched in order to form the electrodes 44, 54 with a width of 1 mm and a spacing of 5 mm, making it possible to obtain an overall transmission of about 85% above 360 nm, while maintaining very satisfactory homogeneity.

In a variant (not shown), the electrodes are in the glass 2, forming a reinforced glass.

The internal faces 22, 32 bear a coating 6″ of phosphor material emitting radiation in the UVA above 350 nm, such as YPO₄:Ce (peak at 357 nm), (Ba,Sr,Mg)₃Si₂O₇:Pb (peak at 372 nm) or SrB₄O₇:Eu (peak at 386 nm).

Of course, other phosphors and a borosilicate glass may be chosen in order to transmit UVA at around 300-330 nm.

Moreover, the external face 31 bears a coating 62 of UV-reflecting material, for example aluminum, in order to increase the transmission and provide protection from the radiation irrespective of the glass chosen for the plate 3.

This UVA lamp 1′″ may be used for example to initiate photochemical processes.

Of course, one or more of the features illustrated in one of the embodiments described above may also be transposed to other embodiments.

Thus, the lamination of the second embodiment may also be used as a variant in the first embodiment.

1-19. (canceled)

20: A flat lamp transmitting radiation in UV (ultraviolet), comprising:
a first dielectric element and a second glass element that are substantially flat and kept substantially parallel to each other and that define an internal space filled with gas capable of emitting the radiation in the UV or of exciting a phosphor material and emitting the radiation in the UV, the phosphor material if present being deposited on one face of the first and/or second element; and
a plurality of pairs of electrodes capable of being at different potentials and of being supplied by an AC voltage, the pairs being associated with the first element and placed outside the internal space,
wherein the first and/or the second element are made of a material that transmits the UV radiation, wherein the first element is a glass element and the electrodes are in a form of bands and/or wires in the first glass element or in another dielectric element associated with the first glass element.

21: The flat lamp transmitting radiation in the UV as claimed in claim 20, wherein the electrodes are covered or integrated at least partly in a dielectric element chosen from the first glass element, another glass element, and/or at least one plastic.

22: The flat lamp transmitting radiation in the UV as claimed in claim 20, wherein the electrodes are placed in a laminated glass that includes the first glass element.

23: The flat lamp transmitting radiation in the UV as claimed in claim 20, wherein the electrodes are placed directly on the first element.

24: The flat lamp transmitting radiation in the UV as claimed in claim 20, wherein the bands are of linear shape, or in a form of interpenetrated combs with a constant spacing between adjacent teeth, or of nonlinear, angled, V-shaped, corrugated or zigzagged form, the spacing between electrodes being kept substantially constant.

25: The flat lamp transmitting radiation in the UV as claimed in claim 20, wherein the electrodes are based on a material transmitting the UV radiation or are arranged and/or configured to allow sufficient overall UV transmission, and wherein the first glass element is made of the material transmitting the UV radiation.

26: The flat lamp transmitting radiation in the UV as claimed in claim 25, wherein the second glass element absorbs the UV radiation.

27: The flat lamp transmitting radiation in the UV as claimed in claim 20, wherein the electrodes are substantially parallel bands and have a width and are spaced apart by a distance, and wherein the ratio of the width to the distance is between 10% and 50%.

28: The flat lamp transmitting radiation in the UV as claimed in claim 20, wherein at least some of the bands are solid bands, or formed from contiguous conducting wires, or parallel wires or braided wires, or from a ribbon or from a coating.

29: The flat lamp transmitting radiation in the UV as claimed in claim 20, wherein at least some of the electrodes are in a form of one or more arrays of essentially elongate conducting features, or conducting lines or conducting wires, substantially straight or corrugated or zigzagged, or organized like a grid, fabric or cloth.

30: The flat lamp transmitting radiation in the UV as claimed in claim 29, wherein the array is defined by a given width of conducting features and a pitch between the conducting features, the pitch is between 5 μm and 2 cm and the width is between 1 μm and 1 mm.

31: The flat lamp transmitting radiation in the UV as claimed in claim 30, wherein the ratio of the width to the pitch is equal to 50% or less.

32: The flat lamp transmitting radiation in the UV as claimed in claim 20, further comprising a material that reflects the UV radiation and that partially or entirely covers one face of the first or second glass element.

33: The flat lamp transmitting radiation in the UV as claimed in claim 20, wherein the material transmitting the UV radiation is chosen from quartz, silica, magnesium fluoride, calcium fluoride, a borosilicate glass, or a glass containing less than 0.05% Fe₂O₃.

34: The flat lamp transmitting radiation in the UV as claimed in claim 20, wherein the phosphor material forms a coating on an internal face of the first glass element and/or on one face of the second glass element.

35: The flat lamp transmitting radiation in the UV as claimed in claim 20, further comprising a coating made of another phosphor material that emits in the visible, associated with the second glass element and placed in a limited peripheral region.

36: The use of the flat lamp transmitting radiation in the UV as claimed in claim 11 in beauty, biomedical, electronics, and food fields.

37: The use of the flat lamp transmitting radiation in the UV as claimed in claim 20 as a tanning lamp, for dermatological treatment, for disinfection or sterilization of surfaces, air or tap water, drinking or swimming pool water, for treatment of surfaces, before deposition of active films, for activating a photochemical process of polymerization or crosslinking type, for drying paper, for analyses on basis of fluorescent material, or for activating a photocatalytic material.

38: A household electrical product incorporating the flat lamp as claimed in claim 20.
